(19) United States
(12) Patent Application Publication
(10) Pub. No.: US 2003/0216801 A1
(43) Pub. Date: Nov. 20, 2003

(54) Transmyocardial implant with natural vessel graft and method

(75) Inventors: Katherine S. Tweden, Mahtomedi, MN (US); Michael Schollmeyer, Maple Grove, MN (US)

Correspondence Address: MERCHANT & GOULD PC, P.O. BOX 2903, MINNEAPOLIS, MN, 55402-0903, US

(73) Assignee: HeartStent Corporation

(21) Appl. No.: 10/150,621

(22) Filed: May 17, 2002

(51) Int. Cl. A61F 2/06

(52) U.S. Cl. 623/1.13

(57) ABSTRACT

A transmyocardial implant includes a hollow rigid conduit having a vessel portion and a myocardial portion. The vessel portion is sized to be inserted into a blood vessel segment taken from a non-coronary artery or vein. The myocardial portion is sized to extend from the vessel portion and through a myocardium into a heart chamber. The conduit has open vessel and myocardial ends on respective ends of the vessel and myocardial portions to define a blood flow pathway within an interior of the conduit between the vessel and myocardial ends. The myocardial portion is formed of a conduit material sufficiently rigid to resist deformation and closure of the pathway in response to contraction of the myocardium. The vessel portion has a radial compliance approximating a radial compliance of the vessel.

### FIELD OF THE INVENTION

This invention pertains to an implant for passing blood flow directly between a chamber of the heart and a coronary vessel. More particularly, this invention pertains to a transmyocardial implant with a non-coronary blood vessel attached to the implant.

### BACKGROUND OF THE INVENTION

Coronary artery disease is the leading cause of premature death in industrialized societies. The mortality statistics tell only a portion of the story. Many who survive face prolonged suffering and disability.

Arteriosclerosis is “a group of diseases characterized by thickening and loss of elasticity of arterial walls.” DORLAND'S ILLUSTRATED MEDICAL DICTIONARY 137 (27th ed. 1988). Arteriosclerosis “comprises three distinct forms: atherosclerosis, Monckeberg's arteriosclerosis, and arteriolosclerosis.” Id.

Coronary artery disease has been treated by a number of means. Early in this century, the treatment for arteriosclerotic heart disease was largely limited to medical measures of symptomatic control. Evolving methods of diagnosis, coupled with improving techniques of post-operative support, now allow the precise localization of the blocked site or sites and either their surgical re-opening or bypass.

The traditional open-chest procedure for coronary artery bypass grafting requires an incision of the skin anteriorly from nearly the neck to the navel, the sawing of the sternum in half longitudinally, and the spreading of the ribcage with a mechanical device to afford prolonged exposure of the heart cavity. If the heart chamber or a vessel is opened, a heart-lung, or cardiopulmonary bypass, procedure is usually necessary.

Depending upon the degree and number of coronary vessel occlusions, a single, double, triple, or even greater number of bypass procedures may be necessary. Often each bypass is accomplished by the surgical formation of a separate conduit from the aorta to the stenosed or obstructed coronary artery at a location distal to the diseased site.

The major obstacles to coronary artery bypass grafting include both the limited number of vessels that are available to serve as conduits and the skill required to effect complicated multiple vessel repair. Potential conduits include the two saphenous veins of the lower extremities, the two internal thoracic (mammary) arteries under the sternum, and the single gastroepiploic artery in the upper abdomen.

Newer procedures using a single vessel to bypass multiple sites have evolved. This technique has its own inherent hazards. When a single vessel is used to perform multiple bypasses, physical stress(e.g.,torsion) on the conduit vessel can result. Such torsion is particularly detrimental when this vessel is an artery. Unfortunately, attempts at using artificial vessels or vessels from other species (xenografts), or other non-related humans (homografts) have been largely unsuccessful. See LUDWIG K. VON SEGESSER, ARTERIAL GRAFTING FOR MYOCARDIAL REVASCULARIZATION: INDICATIONS, SURGICAL TECHNIQUES AND RESULTS 38-39 (1990)

While experimental procedures transplanting alternative vessels continue to be performed, in general clinical practice, there are five vessels available to use in this procedure over the life of a particular patient. Once these vessels have been sacrificed or affected by disease, there is little or nothing that modern medicine can offer. It is unquestionable that new methods, not limited by the availability of such conduit vessels, are needed.

Direct revascularization devices (DRDs) provide an alternative to traditional vein graft bypass operations incorporating harvested vessels. DRDs permit the revascularization of coronary vessels by placement of an artificial conduit between a heart chamber and the coronary vessel, allowing blood flow directly from the heart chamber into a lumen of the vessel. DRDs and methods for implanting such devices are described in U.S. Pat. No. 5,944,019.

DRDs incorporating conduit portions with different degrees of radial compliance are described in currently pending and commonly assigned U.S. patent application Ser. No. 09/304,650. Utilization of varying degrees of radial compliance allows the conduit to have sufficient rigidity within the muscle of the heart wall to prevent collapse while having flexibility more closely matching that of the target vessel.

Further, U.S. Pat. No. 6,250,305 describes the incorporation of natural vessel grafts with artificial conduit DRDs to perform revascularization. The techniques described in the '305 patent allow the connection to the target vessel to be made using a natural vessel graft. While this is a distinct improvement to prior art of vein graft bypass procedures described above, the issue of only having a limited number of suitable vessel for natural graft bypass remains. An approach permitting direct revascularization of coronary vessels incorporating the advantages of artificial conduit DRDs with the advantages of natural vessel grafts is desirable.

### SUMMARY OF THE INVENTION

The present invention relates to an implant for establishing a blood flow path between a heart chamber and a coronary vessel through the myocardium. The implant includes a hollow conduit having a vessel portion and a myocardial portion. The myocardial portion is sized to extend from the vessel portion and through the myocardium to the heart chamber. The myocardial portion is preferably formed of a conduit material sufficiently rigid to resist deformation and closure of the pathway in response to contraction of the myocardium. The vessel portion extends outside the heart wall. In certain embodiments, the vessel portion can have an open structure such as a mesh. In one embodiment, the vessel portion is connected to the coronary vessel by using a relatively short natural graft section that is secured to the open mesh and is also secured to the coronary vessel.

### BRIEF DESCRIPTION OF THE DRAWINGS

FIG. 1 is a side perspective view of a prior art transmyocardial conduit for use with the present invention.

FIG. 2 is a side perspective view of a prior art stent for use with a transmyocardial conduit for use with the present invention, shown elongated to define a smaller diameter.

FIG. 3 is a side perspective view of the prior art stent of FIG. 2, shown shortened to define a larger diameter.

FIG. 4 is a side cross-sectional view of the prior art transmyocardial conduit of FIG. 1 with the stent of FIGS. 2 and 3 mounted about a vessel end.

FIG. 5 is a cross-sectional view of an occluded coronary vessel on a heart wall.

FIG. 6 is a cross-sectional view of the coronary vessel of FIG. 5 with the vessel incised and legated distal the occlusion.

FIG. 7 is a cross-sectional view of the coronary vessel of FIG. 6 with the transmyocardial conduit of FIG. 4 extending through the myocardium.

FIG. 8 is a cross-sectional view of the coronary vessel of FIG. 7 with the transmyocardial conduit connecting a heart chamber with a lumen of the coronary vessel.

FIG. 9 is a perspective view of a second embodiment of a transmyocardial conduit connecting a heart chamber to a lumen of a coronary vessel without a stent about the conduit.

FIG. 10 is a partial cross-sectional view of a third embodiment of a transmyocardial conduit according to the present invention connecting a heart chamber with a lumen of a coronary vessel including a tissue-growth promoting material about the myocardial portion of the conduit.

FIG. 11 is a side perspective view of a portion of a heart wall with a fourth embodiment of a transmyocardial conduit connecting a heart chamber with a lumen of a coronary vessel via an end-to-side anastomosis.

FIG. 12 is a side perspective view of a portion of a heart wall with a fifth embodiment of a transmyocardial conduit connecting a heart chamber with a lumen of a coronary vessel via an end-to-side anastomosis with the conduit at a non-perpendicular angle to the vessel.

### DETAILED DESCRIPTION

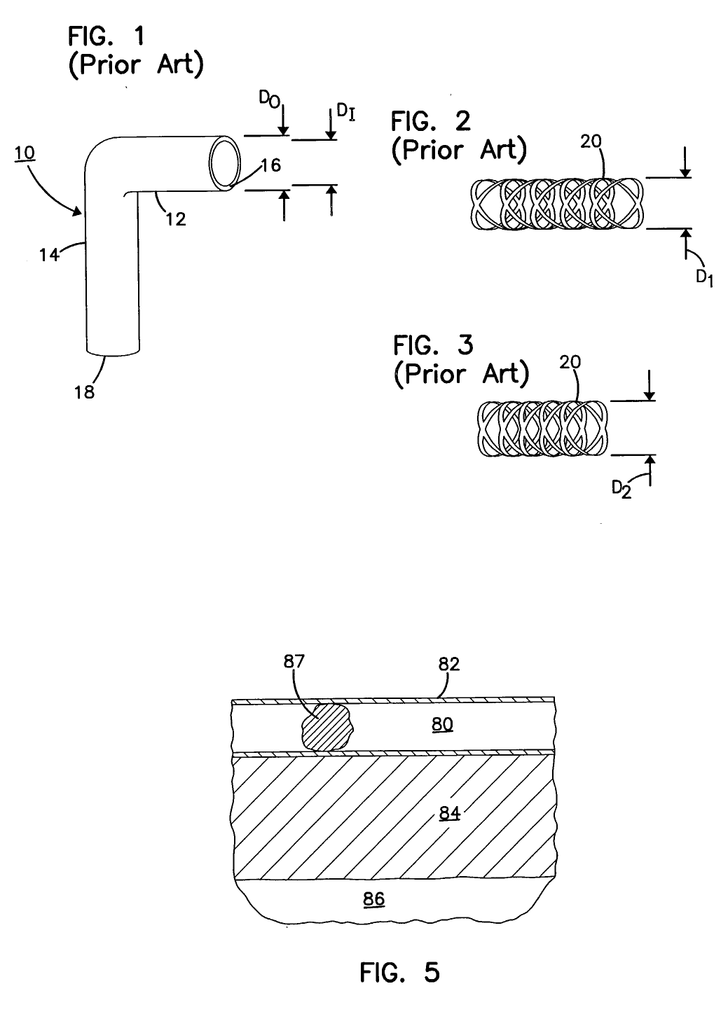

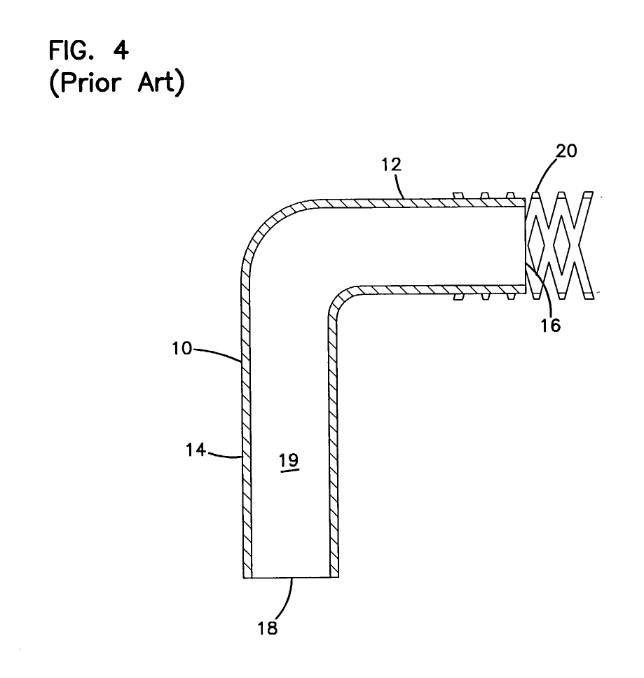

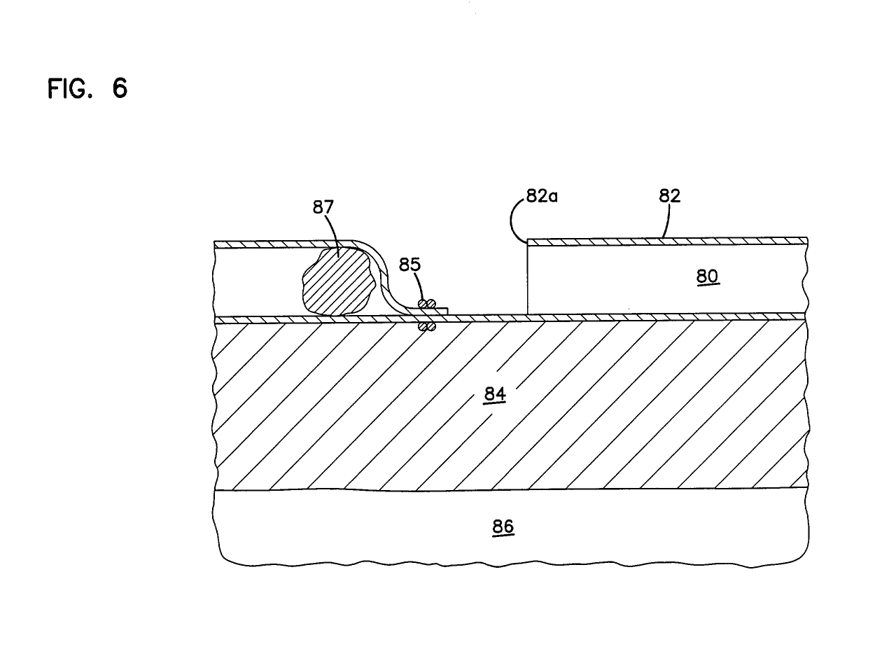

With initial reference to FIGS. 1 through 4, a prior art transmyocardial conduit 10 is shown in the form of an L-shaped rigid tube. In this embodiment, conduit 10 is made of titanium but may be made of any other rigid biocompatible material such as pyrolytic carbon or may be titanium coated with pyrolytic carbon. The material of the conduit 10 is preferably sufficiently rigid to withstand contraction forces of the myocardium. By way of example, conduit 10 will have an outside diameter in the range of about 1 to 4 millimeters and a wall thickness of about 0.25 millimeters.

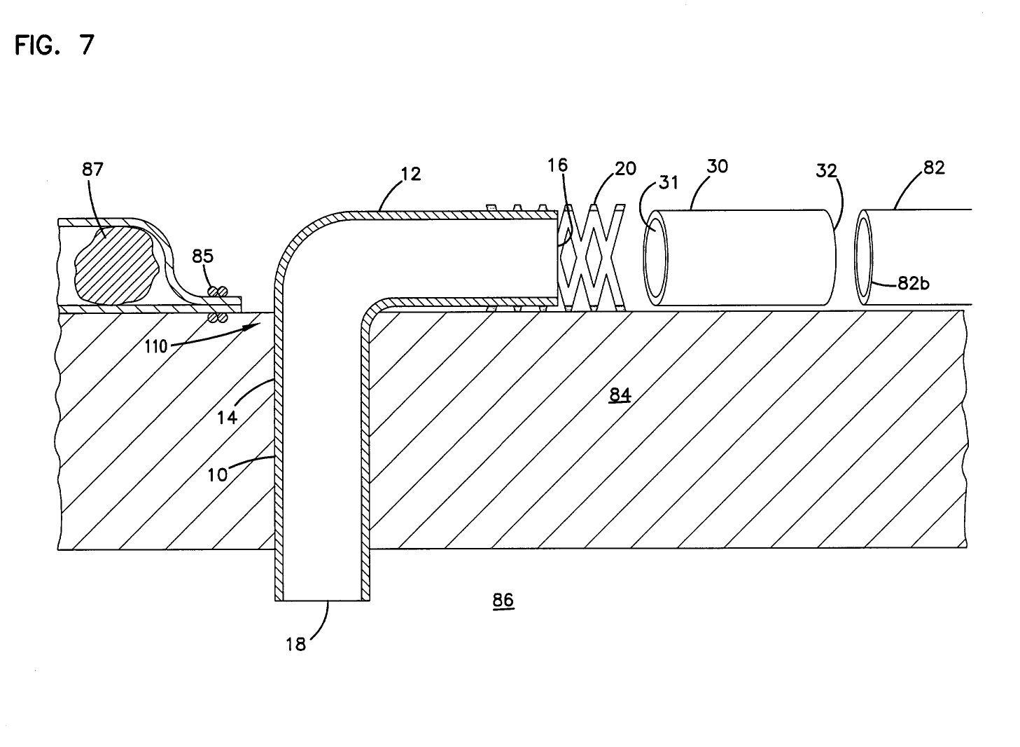

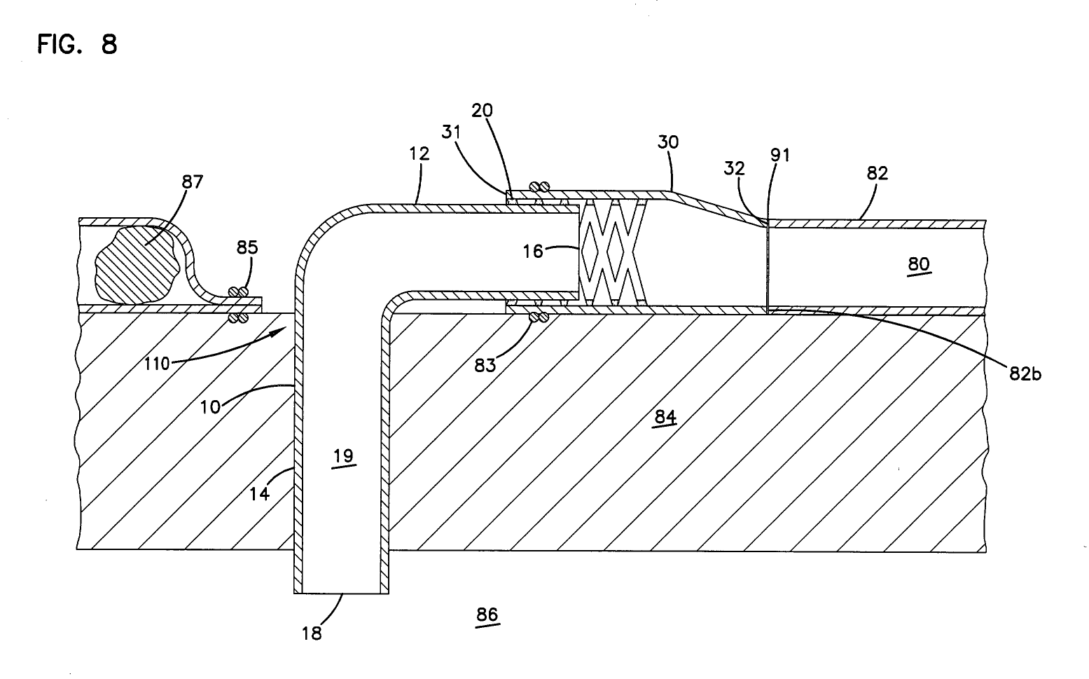

Conduit 10 has a vessel portion 12 including a first open end 16 into an interior 19. Conduit 10 has a myocardial portion 14 extending at an angle to the axis of portion 12 and including a second open end 18. Myocardial portion 14 is sized to extend through the myocardium 84 (as shown in FIG. 7) so that vessel portion 12 is at or near an outer wall of myocardium 84 and open end 18 of myocardial portion 14 protrudes into a heart chamber 86 of a patient's heart.

Conduit 10 may include a stent 20 (shown in FIGS. 2, 3 and 4) which may be a tubular member of lattice formed of biocompatible material. When elongated, stent 20 has an initial diameter D₁ (shown in FIG. 2) which is larger than a conduit outer diameter D₀ (shown in FIG. 1) and further sized for stent 20 to be inserted into lumen 80 of the vessel to be used as a connector. When shortened, stent 20 is expandable to an enlarged diameter D₂ (shown in FIG. 3). It will be appreciated that coronary stents such as stent 20 are commercially available in a wide variety of sizes, shapes, materials and mode of expansion (e.g., self-expanding or balloon expandable). Stent 20 can be any member whose outside dimensions expand to fit within a lumen 80 of a coronary vessel 82 (see FIG. 5) and whose internal dimensions permit insertion of vessel portion 12 within stent 20. Conduit 10 and stent 20 are described in further detail in U.S. Pat. No. 6,053,942, the disclosure of which is incorporated herein by reference.

Referring now to FIGS. 5 through 8, use of a first embodiment of a transmyocardial conduit 10 to revascularize a coronary vessel 82 with an occlusion 87 is shown. Vessel 82 lies on an outer surface of myocardium 84. Occlusion 87 prevents adequate flow of blood to vessel 82 distal to occlusion 87, as shown in FIG. 5. To provide adequate blood flow distal to occlusion 87, vessel 82 is legated distal to an obstruction 87 with sutures 85. An incision is made through the vessel 82 distal to the legating suture 85, as shown in FIG. 6, defining a first distal incised end 82a.

A portion of vessel 82 is dissecting at first incised end 82a to define a second distal incised end 82b. The segment of vessel removed between first incised end 82a and second incised end 82b may be used to form a graft 30 having ends 31 and 32 (see FIG. 7). Alternatively, as discussed below, another source may be available to provide graft 30. For example, in another embodiment, graft 30 is a natural vein segment harvested form within the body of the patient for whom the coronary vessel revascularization is being performed. In a gap defined between occlusion 87 and incised end 82a, a blood flow pathway is formed through myocardium 84 to allow fluid communication with a heart chamber 86. Conduit 10 is placed within the blood flow pathway with myocardial portion 14 extending the myocardium 84 into heart chamber 86, as shown in FIG. 7. Vessel portion 12 of conduit 10 lies along an exterior surface of myocardium 84. Fixed about an end of vessel portion 12 opposite myocardial portion 14 is stent 20.

In FIG. 8, end 31 of graft 30 is positioned about stent 20 and secured by sutures 83 to stent 20. End 32 of graft 30 has been attached to vessel 82 and allows fluid communication between heart chamber 86 and lumen 80 via an interior 19 of conduit 10. The connection between graft 30 and vessel 82 is an end-to-end anastomosis 91.

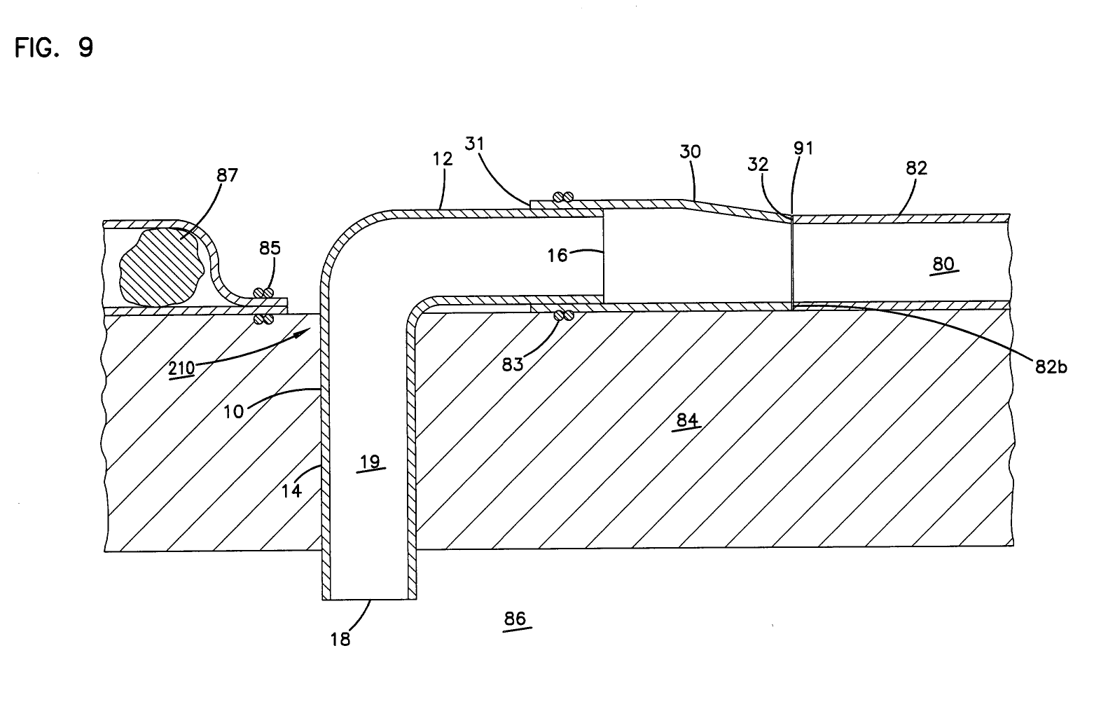

Referring now to FIG. 9, a second embodiment of the present invention is shown. This embodiment is similar to the first embodiment described above with reference to FIGS. 5 through 8. In the second embodiment, graft 30 is connected directly to vessel end 12 of conduit 10 without stent 20 interposed between and is secured to conduit 10 with sutures 83. All other elements of the first embodiment are included in the second embodiment.

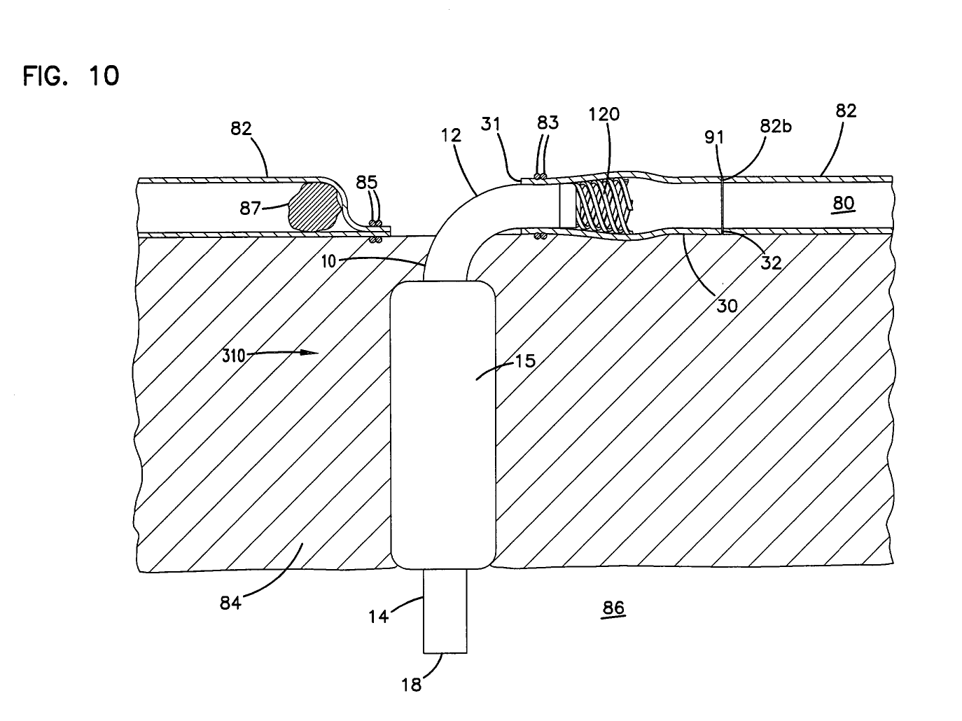

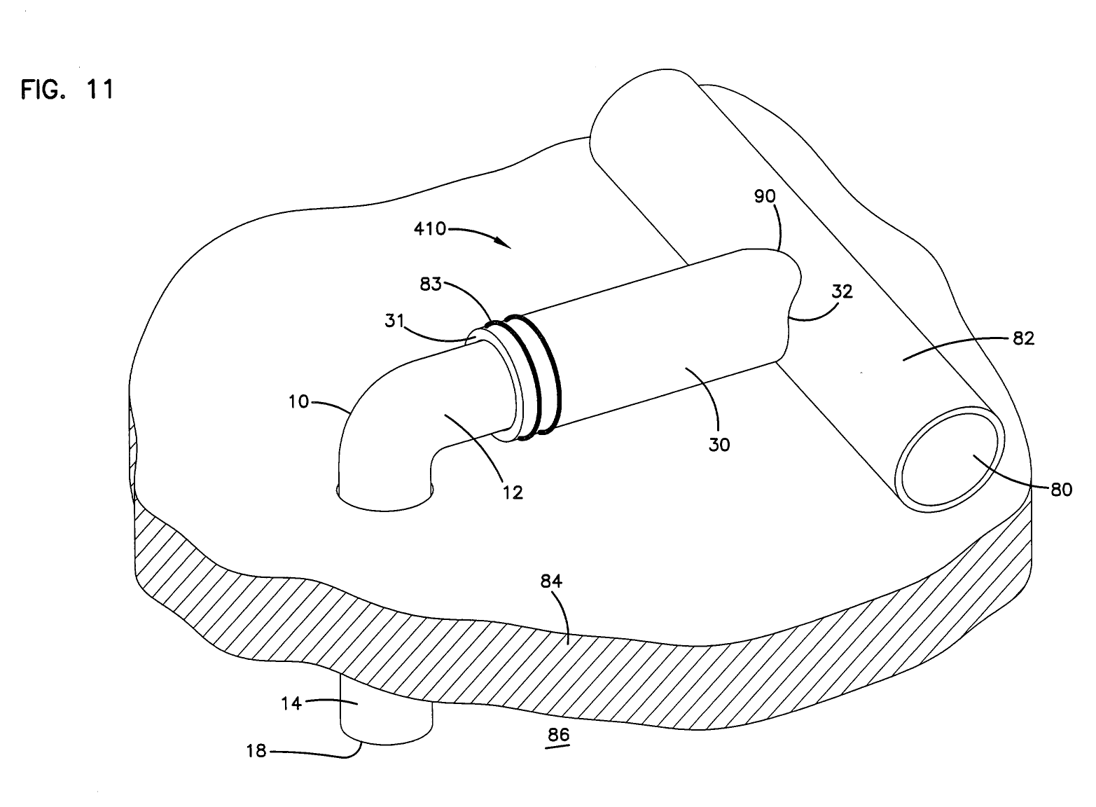

Referring now to FIG. 10, a third embodiment of a transmyocardial conduit for revascularizing a coronary vessel is shown. This embodiment is similar to the first embodiment detailed above with the additional of a sleeve 15 made of a tissue-growth inducing material such as polyester about myocardial portion 14. The use of such sleeves about myocardial implants to anchor the implants within the myocardium is discussed in further detail in U.S. Pat. No. 5,984,956, the disclosure of which is incorporated herein by reference.

In place of stent 20, conduit 10 in FIG. 10 includes a compliant sleeve 120 about which is mounted graft 30. Complaint sleeve 120 has a degree of radial compliance which is adapted to match the radial compliance of graft 30. Further description of the use of sleeve 120 with radial compliance matched to the compliance of the vessel into which the sleeve extends is found in commonly-assigned pending U.S. patent application Ser. No. 09/304,650, the disclosure of which is incorporated herein by reference.

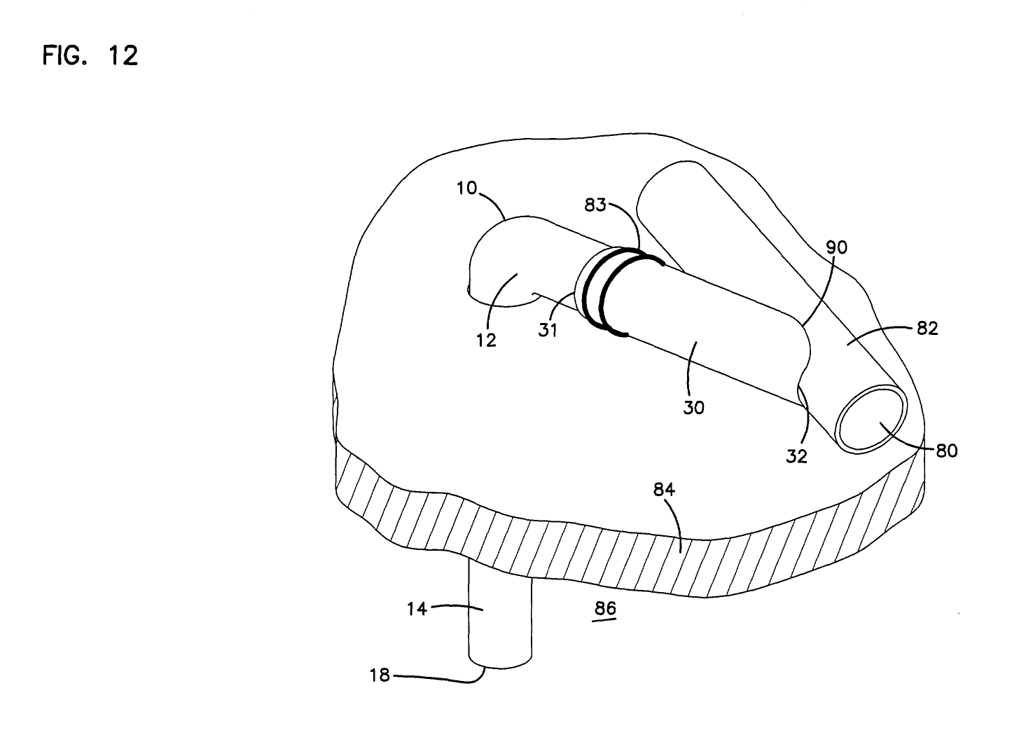

Referring now to FIG. 11, a further embodiment of a transmyocardial conduit for revascularizing a coronary vessel is shown. In this embodiment, conduit 10, with or without stent 20, provides fluid communication between heart chamber 86 and lumen 80 via graft 30 which is connected to vessel 82 with an end-to-side anastomosis 90. This embodiment does not require vessel 82 to be incised or dissected. However, since vessel 82 is not being incised or dissected, an alternative source for graft 30 within the patient's body will need to be found. Following a standard method of coronary artery bypass surgery, a portion of a suitable blood vessel such as the internal mammary artery may be available from segments of the artery not required for the bypass procedure. Alternatively, other vessels may be used as a source for graft 30, such as the radial artery, the lesser saphenous vein, an arm vein, the gastroepiploic artery, the inferior epigastric artery or other vessels of suitable size.

Conduit 10 is placed by inserting second portion 14 through myocardium 84 with open end 18 in communication with left ventricle 86. First portion 12 is inserted into enlarged stent 20 (See FIGS. 1, 5). An embodiment of a method of placing an implantable conduit between a chamber of the heart and a coronary vessel is described in detail in U.S. Pat. No. 5,755,682, the disclosure of which is hereby incorporated by reference.

In one embodiment of the invention, following a standard method of coronary artery bypass surgery, the surgeon may have a portion 30 of a suitable blood vessel such as the internal mammary artery. Depending on the availability of vessels and the technique preferred by the surgeon other vessels may be used such as the radial artery, the lesser saphenous vein, an arm vein, the gastroepiploic artery or the inferior epigastric artery. Portion 30 has two ends 31, 32. The surgeon then takes portion 30 of this residual vessel (for example, the internal mammary artery) and slides end 31 over the stent 20. (See FIG. 5) Eend 32 of the portion 30 is connected to end 82b of the ligated coronary artery 82 by methods well known to those with skill in the art. The surgically connected structure consisting of the stent 20, the piece of blood vessel 30 and the coronary artery 82 is then stabilized on the myocardium 84.

This may be the preferred embodiment of the present application in that it allows a more efficient and complete usage of harvested vessels during bypass procedures. Using the present invention in conjunction with standard vein graft bypass procedures will permit multiple bypasses to be created with a single harvested vessel by utilizing pieces of the native vessel that otherwise would have been discarded. In this way, patients requiring additional bypass procedures at a future date will still have usable vessels for traditional bypass procedures. Alternatively, for patients who have no remaining vessels suitable for traditional bypass procedures, the present method offers an approach which utilizes vessels not otherwise considered usable for bypass.

In another embodiment of the invention shown in FIG. 11, conduit 10 is implanted by inserting second portion 14 through myocardium 84 with open end 18 in communication with left ventricle 86. End 31 of portion 30 is attached to first portion 12 of conduit 10 either directly or utilizing a stent 20 depending on the preference of the surgeon. End 32 is then anastomosed to the selected coronary artery 82 via an end to side anastomosis 90.

FIG. 12 shows a similar embodiment to that shown in FIG. 11, with the difference being that conduit 10 forms an end-to-side anastomosis 90 with vessel 82 at an angle. The angle of anastomosis 90 is angled to bias flow out of conduit 10 in the direction of normal blood flow within vessel 80.

From the foregoing, the invention has been described in a preferred embodiment. Modifications and equivalents of the disclosed concepts are intended to be included within the scope of the claims.

What is claimed is:

1. A method revascularizing a coronary vessel of a patient comprising:
inserting a hollow conduit through a heart wall so that a first end of the conduit extends into a heart chamber and a second end of the conduit extends beyond the heart wall;
connecting the second end of the conduit with a first end of a natural vessel graft wherein the first end of the natural vessel graft is outside the heart wall; and
attaching a second end of the natural vessel graft to the coronary artery.

2. The method of claim 1, wherein the coronary vessel includes an occlusion which at least partially blocks a flow of blood within the coronary distal the occlusion, the connection between the natural vessel graft and the coronary vessel is distal the occlusion and a blood flow from the second end of the natural vessel graft into the coronary vessel is directed away from the occlusion.

3. The method of claim 1, wherein a stent is attached to the second end of the conduit and the natural vessel graft is attached to the stent.

4. The method of claim 1 wherein the coronary vessel is a coronary artery.

5. The method of claim 1, wherein the heart chamber is a left ventricle.

6. The method of claim 1, wherein the natural vessel graft is from a non-coronary vessel within the patient.

7. The method of claim 6, wherein the natural vessel graft is selected from one of an internal mammary artery, a lesser saphenous vein, a gastroepiploic artery, an inferior epigastric artery, and an arm artery.

8. The method of claim 2, wherein the conduit is inserted through the heart wall within the coronary vessel.

9. The method of claim 2, wherein the coronary vessel is incised distal the occlusion forming a first end adjacent to the occlusion and a second end, the first end of the coronary vessel being closed and the second end of the natural vessel graft is attached to the second end of the coronary vessel with an end to end anastomosis.

10. The method of claim 2, wherein the conduit is inserted through the heart wall offset from the coronary vessel and the second end of the natural vessel graft attached to the coronary vessel with an end to side anastomosis.

11. The method of claim 10, wherein the end to side anastomosis is at an angle with respect to an axis of flow of the coronary vessel.

12. The method of claim 11, wherein the angle is oriented to direct blood flow from the second end of the vessel graft in the direction of blood flow in the coronary vessel.

13. An implant for revascularizing a coronary vessel comprising:
a hollow conduit with a first portion with a first end and a second portion with a second end, the first portion adapted to be positioned with a myocardium and extend into a heart chamber, the second portion extending outside the myocardium;
a stent attached to the second end of the hollow conduit;
a natural vessel graft with a first end attached to the stent so that no portion of the natural vessel graft is within the myocardium when the first portion of the conduit is placed with the myocardium, and a second end adapted to be attached to the coronary vessel;
wherein the implant is adapted to provide fluid communication between the heart chamber and the coronary vessel when the first portion is placed within the myocardium extending into the heart chamber and the second end of natural vessel graft is attached to the coronary vessel.

14. The implant of claim 13, wherein the coronary vessel is a coronary artery.

15. The implant of claim 13, wherein the heart chamber is a left ventricle.

16. The implant of claim 14, wherein the natural vessel graft is from a non-coronary vessel.

17. The implant of claim 16, wherein the natural vessel graft is selected from one of an internal mammary artery, a lesser saphenous vein, a gastroepiploic artery, an inferior epigastric artery, and an arm artery.

18. The implant of claim 13, wherein the second end of the natural vessel graft is attached to the coronary vessel by an end to side anastomosis.

19. The implant of claim 13, wherein the second end of the natural vessel graft is attached to the coronary vessel by an end to end anastomosis.

20. The implant of claim 19, wherein the end to side anastomosis is at an angle with respect to an axis of flow of the coronary vessel.

21. The implant of claim 20, wherein the angle is oriented to direct blood flow from the second end of the vessel graft in the direction of blood flow in the coronary vessel.
